(19) United States
(12) Patent Application Publication
(10) Pub. No.: US 2018/0064835 A1
(43) Pub. Date: Mar. 8, 2018

(54) A CONTAINER, METHOD AND SYSTEM FOR STERILISING A MEDICAL DEVICE

(71) Applicant: DEPUY IRELAND UNLIMITED COMPANY, County Cork (IE)

(72) Inventor: DUNCAN YOUNG, MELBOURN (GB)

(21) Appl. No.: 15/565,340

(22) PCT Filed: Apr. 5, 2016
(86) PCT No.: PCT/EP2016/057403

§ 371 Date: Oct. 9, 2017

(30) Foreign Application Priority Data
Apr. 15, 2015 (GB) 1506351.4

(51) Int. Cl. A61L 2/18; B65B 31/04; B65B 55/18; B65B 51/14

(52) U.S. Cl. CPC A61L 2/18; B65B 31/042; A61L 2202/181; B65B 51/148; A61L 2202/24; B65B 55/18

(57) ABSTRACT

The present invention relates to a container, a method and a system for sterilising a medical device. The invention is particularly, although not exclusively, for use with orthopaedic medical devices, such as surgical instruments used in orthopaedic surgical procedures.

The present invention relates to a container, a method and a system for sterilising a medical device. The invention is particularly, although not exclusively, for use with orthopaedic medical devices, such as surgical instruments used in orthopaedic surgical procedures.

Any surgical procedure typically requires a number of instruments. Orthopaedic surgery typically requires a large number of instruments during the surgical procedure. The instruments are provided in one or more trays with each tray containing several instruments. The trays are then sterilised with the instruments in place and sealed or wrapped before they are required during surgery. The internal contents of the instrument tray are often not visible through the sterile wraps which means that a health practitioner is not able to check the contents of the instrument set without opening it. If the seal is broken, for example to check the contents, the sterility of all of the instruments within the set will be compromised even if some of them are not ultimately used in surgery. It is therefore necessary to resterilise the entire instrument set before it can be reused.

In practice, the instrument set can be stored in a variety of places and it is then lifted, carried and transported by many people before finding itself in the operating theatre prior to the surgical procedure taking place. This increases the risk that the seals could be damaged for example when lifted off a wire storage shelf.

While the sterility of the instrument set may be very good at the point at which it leaves the autoclave it is understood that there are inherent risks in whether the set is sterile when it reaches the operating theatre. The actual sterility of the set when it reaches the operating theatre is determined by how well the validated sterilisation method has been followed and how carefully the package has been handled. The sterility of the set cannot be verified by the health practitioner prior to opening the health practitioner can only check that integrity of the seal and the wrapping: a breach of either would indicate a high risk of sterility breach.

According to a first aspect of the present invention, there is provided a container for a medical device, the container comprising a first wall and a second wall defining an interior of the container, at least one of the first wall and the second wall being flexible, the medical device being housed within the interior of the container, the container having had suction applied to the interior of the container and sealed such that at least one of the first wall or second wall is configured to encase the medical device and in which the interior of the container has an amount of a liquid sterilising agent contained therein.

The presence of the liquid sterilising agent acts to sterilise the medical device. The medical device may remain in contact with any liquid sterilising agent that remains within the container after the air has been extracted. The use of a liquid sterilising agent is advantageous becomes any leakage of the agent will be indicative to the health practitioner that the sterility of the container has been breached. Furthermore, a health practitioner will be aware that any liquid surrounding the medical device is a sterilising agent which contributes to the confidence in the sterility of the device. This is unlike previously where the presence of liquid could be indicative of contamination.

The liquid sterilising agent may comprise an agent with microbicidal, and/or fungicidal, and/or virucidal, and/or sporicidal properties. The sterilising agent may contain one or more of glutaraldehyde, iodine, povidone-iodine, an alcohol (for example ethanol), trichlorophenol, sodium hypochlorite, peracetic acid, and 5-chloro-2-(2,4-dichlorophenoxy)phenol (triclosan). The liquid sterilising agent, for example the anti-microbial agent, may be provided as an aqueous solution.

A neutralising agent can be placed in the container with the liquid sterilising agent.

The medical device can be any instrument, apparatus, appliance, material or other article, whether used alone or in combination which is intended to be used in animals, including human beings, for the purpose of diagnosis, prevention, monitoring, treatment or alleviation of a disease, pathology, injury or handicap.

Orthopaedic medical devices include, but are not limited to, devices used in joint replacement or fracture management. Joint replacement products include devices or prostheses for hip, knee, ankle, shoulder, elbow, wrist, and finger arthroplasty procedures. Fracture management products include a wide variety of devices including wires, pins, screws, plates, spinal fixation devices, and artificial ligaments.

The container may further include an opening connecting the interior of the container to an exterior of the container through which a medical device can be inserted.

The opening may be configured to be connectable to an air extractor such as a vacuum pump so that the suction can be applied to the interior of the container.

The container may further include a zip fastener at the opening. Alternatively, the opening can be heat sealed.

The container may include at least one port connecting the interior of the container to an exterior of the container and configured to be connectable to an air extractor such as a vacuum pump so that the suction can be applied to the interior of the container.

The at least one port may be an aperture formed in the container. The at least one port may include a removable tube.

The first wall and the second wall may be sealed together with the at least one port provided in a junction between the sealed first wall and second wall.

At least one of the first and second walls may comprise a transparent or translucent material. In this way the medical device in the container is visible and, as such, the contents of the container can be checked without needing to open the packaging.

The first wall and/or the second wall may comprise a flexible material having multiple layers.

Both the first and second walls may be made of a transparent/translucent material. Both of the first and second walls may be flexible.

The first wall and the second wall may be rectangular in shape, sealed along three sides with the opening provided in a fourth side.

The container may include a carry handle.

By using the container of the invention the medical device will remain generally constrained in a fixed position. This has particular advantages during the transit or storage of a plurality of surgical instruments, such as an instrument set. The fixed positioning of the instruments relative to one another enables the instruments to be placed in the container in accordance with a defined arrangement. This enables the health practitioner to easily check that a full set of the instruments are included within the container. The defined arrangement of the instruments also aids in the selection of the instruments from the container during surgery.

In order to facilitate the arrangement of the surgical instruments in a defined order, the interior of the container may further include a template on which a plurality of surgical instruments can be arranged in the container in accordance with the template.

The template may be two-dimensional or three-dimensional. The template may indicate a preferred position of the surgical instruments within the container. This can be indicated graphically or with alphanumeric characters.

The container may be placed in a second container after sealing the opening. The second container may have first and second walls defining an interior of the second container, and an opening, the container being contained within the interior of the second container. The second container may include a sterilising agent, for example a liquid sterilising agent.

According to another aspect of the present invention, there is provided a method of sterilising a medical device, the method including the steps of:
providing a container for the medical device, the container having a first wall and a second wall defining an interior of the container, at least one of the first wall and second wall being flexible, the container further including an opening connecting the interior of the container to the exterior of the container;
placing the medical device into the interior of the container;
introducing a liquid sterilising agent into the interior of the container;
extracting air from the container; and
sealing the container.

The medical device may be placed into the interior of the container through the opening. The sterilising agent may be inserted into the container via the opening. The air may be extracted via the opening.

The container may be sealed by closing the opening.

The container may further include a port connecting the interior of the container to the exterior of the container, the sterilising agent being inserted into the container via the port.

The air may be extracted from the container via the port.

The opening may be closed after the medical device is placed into the interior of the container and the container is sealed by closing the port.

The port may include a one-way valve, a faucet or finger-operated valve. The port may be a removable port.

The port may be configured for coupling to an air extractor, the air extractor being configured to extract air from the container. The air extractor may be a vacuum pump.

The first and second walls may be sealed together with the port provided in a junction between the sealed first and second walls. The first and second walls may be provided integrally. Alternatively, the port may be an aperture formed in the container.

The at least one of the first and second walls may be made of a translucent or transparent material.

The air may be extracted to form a vacuum within the interior of the container by means of an air extractor, for example a vacuum pump.

Prior to extracting the air from the container, the method may include the step of dispersing the sterilising agent within the container by manipulating the container. In this way, contact between the sterilising agent and the medical device is improved. The manipulation may be effected by shaking or moving the container, for example by hand, to ensure that the sterilising agent makes contact with as much of the surface area of the medical device as possible.

Alternatively, the container can be angled to disperse the sterilising agent.

The method may be directed to the sterilisation and storage of a plurality of surgical instruments. In some constructions of the method, the method may further include the step of placing a template underneath the container and then arranging the surgical instruments within the container in accordance with the template. In some other constructions of the method, the method may further include the step of placing a template in the interior of the container before placing the surgical instruments in the sterilisation and then arranging the surgical instruments within the container in accordance with the template.

The method may also be directed to the sterilisation and storage of a plurality of modular components.

The opening of the container may be closed by heat sealing. Alternatively, the container may include a zip fastening mechanism at the opening and the opening being closed by closing the zip fastening.

The air may be extracted by means of a vacuum pump.

The container may be placed in a second container after sealing the opening.

A neutralizing agent may be placed in the container with the sterilising agent.

In accordance with another aspect of the present invention, there is provided a method of sterilising a medical device, the method including the steps of: providing a container for a medical device, the container having a first wall and a second wall defining an interior of the container, at least one of the first wall and second wall being flexible, the container further including an opening and at least one port connecting the interior of the container to the exterior of the container; placing the medical device into the container through the opening; closing the opening; introducing a liquid sterilising agent into the container through the at least one port; extracting air from the container via the at least one port; and sealing the at least one port.

In accordance with another aspect of the present invention, there is provided a container for a plurality of medical devices, the container comprising a first wall and a second wall defining an interior of the container, at least one of the first wall and the second wall being flexible, the plurality of medical devices being arranged on a template located within the interior of the container, the container having had suction applied to the interior of the container and sealed such that at least one of the first wall or second wall is configured to encase the plurality of medical devices and in which the interior of the container has an amount of a sterilising agent contained therein.

Further benefits of the invention include that the packaging of the container is robust and it becomes obvious to an observer if the package has been punctured as there is leaking of the liquid sterilising agent from the container. Health practitioners will be aware that any liquid surrounding the instruments is the liquid sterilising agent which contributes to the confidence in the sterility of the instruments. This is unlike previously where the presence of liquid could be indicative of contamination.

In addition, any ingress of air from a rupture in the vacuum seal would cause the medical device to slump to the bottom of the container.

Furthermore, the container is cheap to manufacture, using known packaging materials and processes. It is low weight and the actual content of the container can be customisable as required by the surgeon, the hospital or in accordance with the needs of the patient. As the surgical instruments can be held in an arrangement and spaced apart, they are individually inspectable.

According to a further aspect of the invention there is provided a pack of surgical instruments, the pack comprising a container for the surgical instruments, the container having a first wall and a second wall defining an interior of the container, at least one of the first wall and the second wall being flexible, the surgical instrument components being housed within the interior, the interior of the container having an amount of a sterilizing agent contained therein, the container having had suction applied to the interior of the container and sealed such that at least one of the first wall or second wall is configured to encase the surgical instruments such that the surgical instruments are restrained within the container when sealed and remain in a fixed arrangement relative to each other.

Optionally, the container further includes an opening connecting the interior of the container to an exterior of the container through which the surgical instruments can be inserted.

Optionally, the container further includes a zip fastener at the opening.

Optionally, the opening is configured for applying the suction to the interior of the container.

Optionally, the container further includes at least one port connecting the interior of the container to an exterior of the container. The at least one port includes a removable tube. The at least one port may be an aperture formed in the container.

Optionally, the first wall and the second wall are sealed together with the at least one port provided in a junction between the sealed first wall and second wall. The first and second walls may comprise a transparent or translucent material. The first wall and/or the second wall may comprise a flexible material having multiple layers.

Optionally, the sterilization container may include a carry handle.

The pack may further include a template in the interior of the container, the surgical instrument components being placed in the sterilization container in accordance with the template.

The sterilizing agent comprises an agent with microbicidal, and/or fungicidal, and/or virucidal and/or sporicidal properties. The sterilizing agent may be water, heated to form superheated water vapour which acts to sterilize the surgical instruments.

According to a further aspect of the invention there is provided a method of sterilizing surgical instruments, the method including the steps of:
providing a sterilization container for surgical instruments, the sterilization container having a first wall and a second wall defining an interior of the sterilization container, at least one of the first wall and second wall being flexible, the sterilization container further including an opening connecting the interior of the sterilization container to the exterior of the sterilization container;
placing the surgical instruments into the interior of the sterilization container;
inserting a sterilizing agent into the interior of the sterilization container;
extracting air from the sterilization container; and
sealing the sterilization container.

The surgical instruments may be placed into the interior of the sterilization container through the opening. They may be inserted into the sterilization container via the opening. The air may be extracted via the opening. The sterilization container may be sealed by closing the opening.

Optionally, the sterilization container further includes a port connecting the interior of the sterilization container to the exterior of the sterilization container, and the sterilizing agent is inserted into the sterilization container via the port. In some examples of the method, the opening is closed after the surgical instruments are placed into the interior of the sterilization container, the air is extracted from the sterilization container via the port, and the sterilization container is sealed by closing the port.

The sterilizing agent may comprise an agent with anti-microbial properties. The sterilizing agent may be water, and the method includes the step of heating the sterilization container to form superheated water vapour which acts to sterilize the surgical instruments.

The method may further include the step of placing a template underneath the sterilization container before placing the surgical instruments in the sterilization container, the surgical instrument components being placed in the sterilization container in accordance with the template.

The method may further include the step of placing a template in the interior of the sterilization container before placing the surgical instruments in the sterilization container, the surgical instrument components being placed in the sterilization container in accordance with the template.

The method may further include the step of dispersing the sterilization agent within the sterilization container.

In some examples of the method, the opening is closed by heat sealing.

In some examples of the method, the sterilization container may include a zip fastening mechanism at the opening and the opening is closed by closing the zip fastening.

Optionally, the sterilization container is placed in a second container after sealing the opening.

According to a further aspect of the invention there is provided a system for sterilizing surgical instruments, the system comprising: a sterilization container having a first wall and a second wall defining an interior of the sterilization container, at least one of the first wall and second wall being flexible, the sterilization container further including an opening connecting the interior of the sterilization container to the exterior of the sterilization container; a vacuum pump; and a flexible tube for coupling the vacuum pump to the interior of the sterilizing container via the opening.

Optionally, the system further includes a second container having first and second walls defining an interior of the second container, and an opening, the second container being configured to contain the sterilization container within the interior of the second container.

Optionally, the system further includes a storage rack for storing the sterilization container thereon.

The invention will now be described by way of example only with reference to the accompanying figures of which:

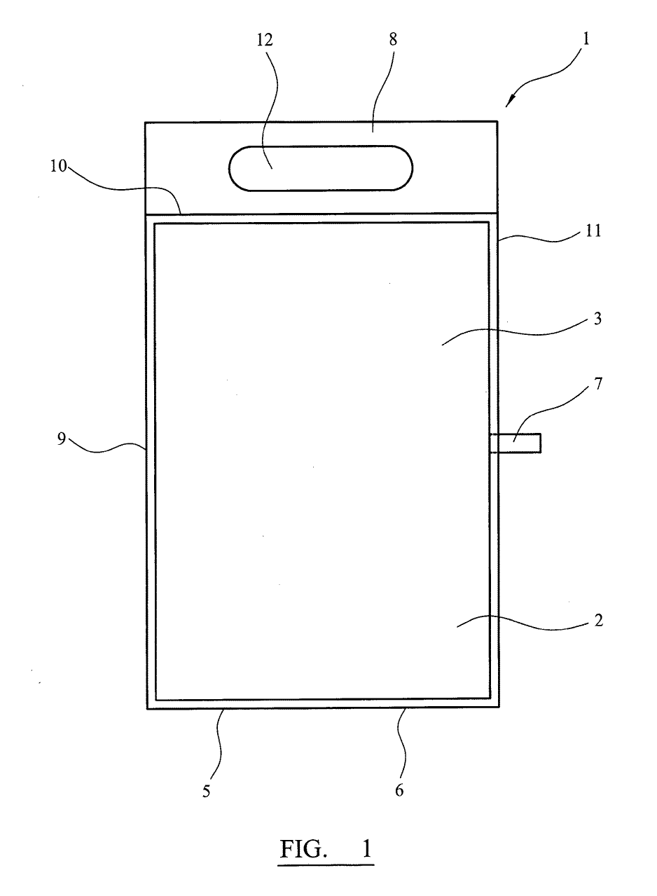

FIG. 1 is a schematic representation of the container of the present invention;

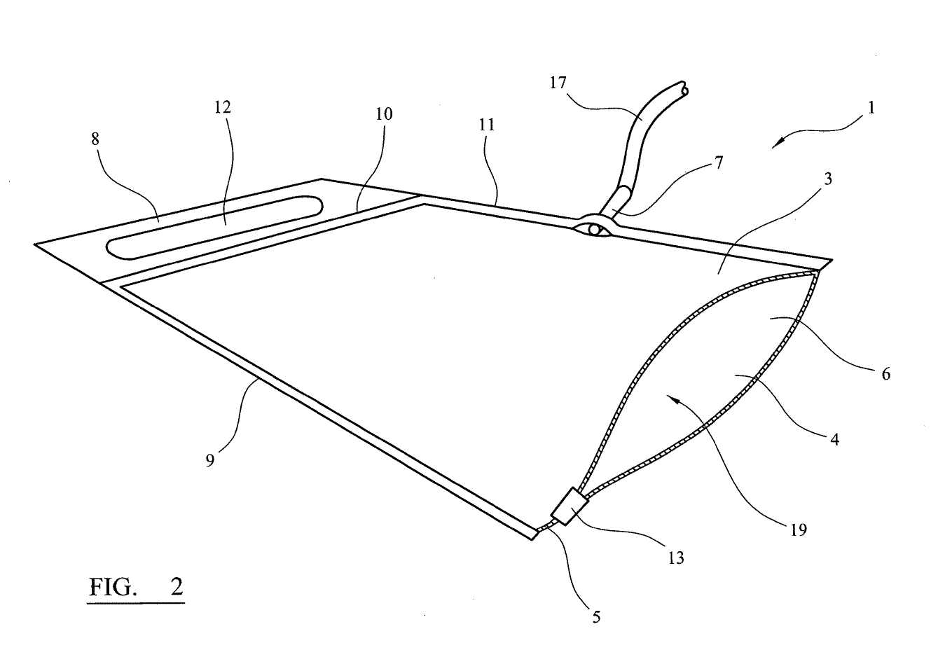

FIG. 2 is a schematic perspective representation of the container having a zip fastener;

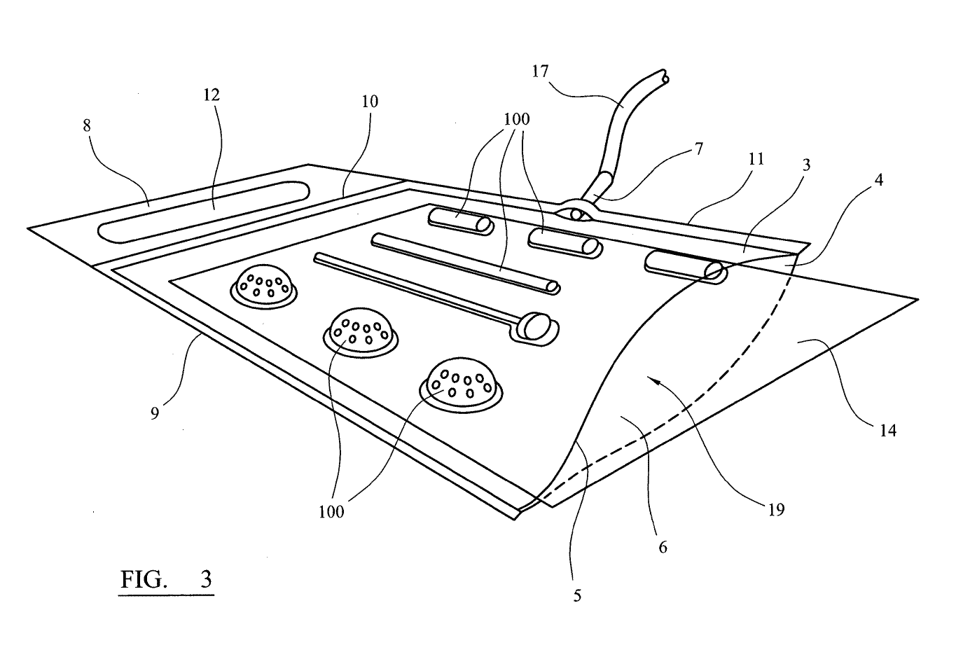

FIG. 3 is a schematic perspective representation of the container during placement of surgical instruments;

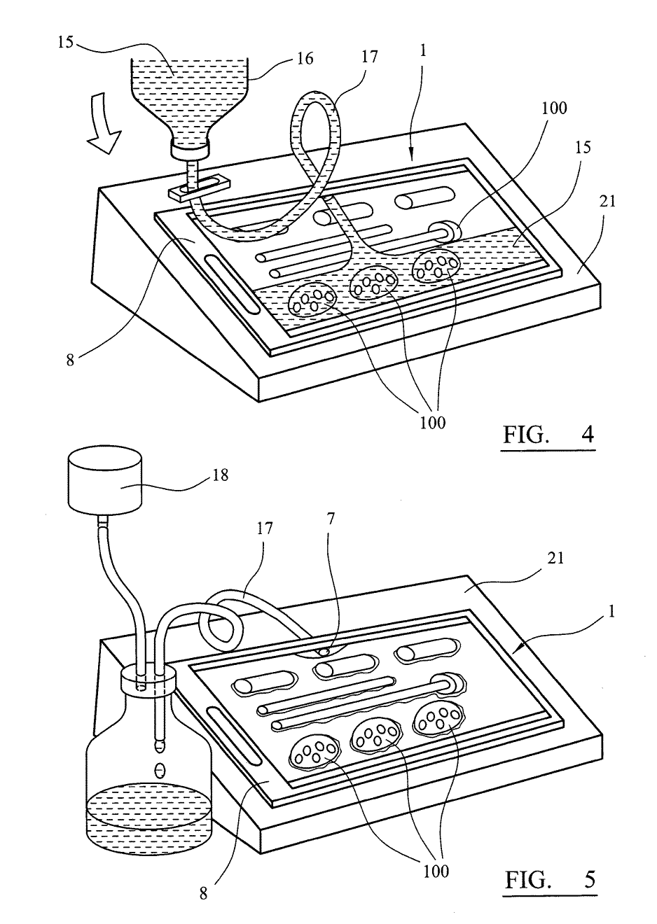

FIG. 4 is a schematic illustration showing the container being filled with a liquid sterilising agent;

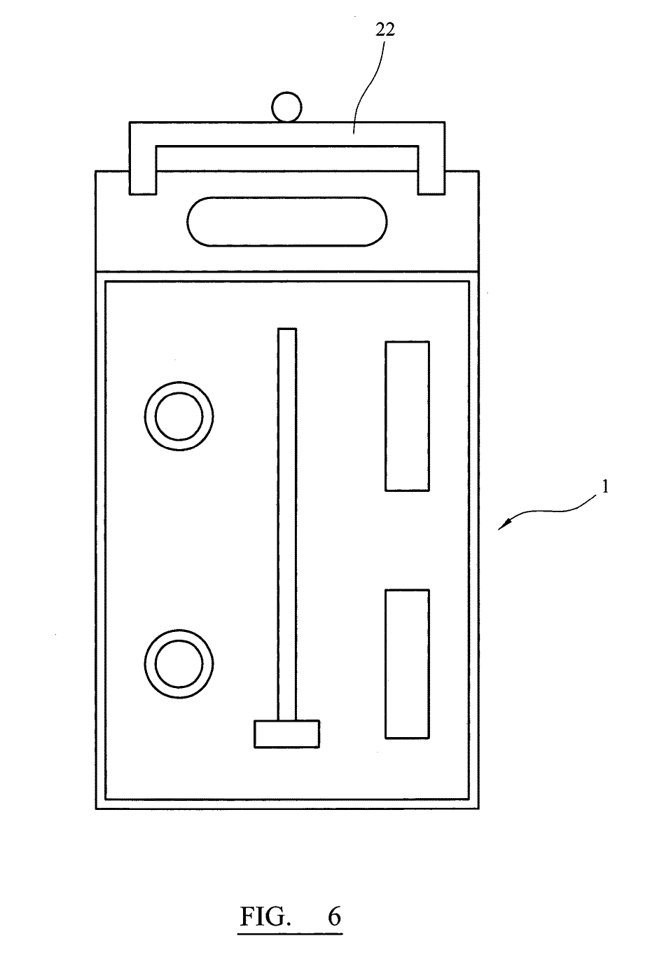

FIG. 5 is a schematic illustration of the container with air and an amount of the liquid sterilising agent being evacuated; and

FIG. 6 is a schematic illustration of the container being stored prior to collection for use in a surgical procedure.

A container 1 for use in sterilising surgical instruments 100 comprises a transparent plastic bag 2. In this embodiment, the bag 2 is rectangular in shape, although other shapes could be used.

The bag 2 comprises a first wall 3 and a second wall 4 sealed around three edges 9, 10, 11 with a fourth side 5 being unsealed before use and defining an opening 6. The first wall 3 and second wall 4 define an interior 19 of the bag 2.

A port 7, in the form of a flexible plastic tube, is provided along one of the sealed edges.

The port 7 provides an inlet/outlet as will be described in further detail below. As alternative, the port 7 can be in the form of a simple aperture formed by a lack of a seal without a flexible plastic tube.

In this embodiment, one or both the first wall 3 and the second wall 4 are made of a flexible, transparent, or translucent, material so that the contents of the bag 2 are visible to a user. The material might be a polymer such as a PVC, a polyethylene, a cellulose material such as a cellophane, a polyester such as polyethylene terephthalate, a nylon or a combination, for example in a blend or layered/laminated structure. The material used forms a gas barrier and a liquid barrier.

The material could be coloured or clear. It could be partially transparent i.e. in a pattern or with opaque areas for labelling. The material can be single layer, or multiple layers i.e., two or more layers. An example of the material that would be suitable would be that used to make bags sold under the FoodSaver® brand by Sunbeam Products Inc. Such bags are five layer polyethylene with an outer layer of nylon. These can be subject to heating through, for example, immersion in boiling water or in a microwave.

In an alternative, one of the first wall 3 and the second wall 4 could be made of a more rigid material, while the other of the first wall 3 and second wall 4 could be of a flexible material.

A carry handle 8 is formed along one 10 of the sealed edges. In the embodiment described herein, the carry handle 8 is formed by forming an elongate aperture 12 in a portion of flexible material formed integrally with the bag 2 at the one edge 10 so that the carry handle 8 is integral with the bag 2. As an alternative, the carry handle could be provided separately of any suitable configuration which would enable the bag 2 to be held in the hand, or suspended on a trolley or inside a carry case.

In an alternative embodiment, the opening 6 can include a zip fastening mechanism 13.

The bag 2 is used to store and sterilise surgical instruments as described below.

Prior to containment of the surgical instruments 100, the bag 2 is unsealed at the opening 6.

Soiled instruments 100 are returned for sterilisation from the operating room in caddies. In accordance with usual procedures, the surgical instruments 100 are hand-rinsed and inspected for damage and dismantled. The surgical instruments 100 are then decontaminated and disinfected in appropriate reprocessing equipment, such as a washer-disinfector, in which they are washed, rinsed and dried in accordance with usual (regulated) protocols and procedures.

After the surgical instruments 100 are washed, rinsed and dried, an operator assembles any surgical instruments 100 as required, inspects each surgical instrument 100 and provides a checklist of the surgical instruments 100 to be stored in the bag 2. Their function is usually checked and they are lubricated as required.

Once the surgical instruments 100 are assembled and checked, the operator places the surgical instruments 100 into the bag 2 via the opening 6.

The surgical instruments 100 are laid out in an appropriate arrangement so that each of the surgical instruments 100 are clearly visible through the bag 2.

To facilitate the correct placement of the instruments, the bag 2 is laid on top of a two-dimensional, or three-dimensional, layout template 14. Alternatively, the template 14 can be placed inside the bag 2.

A three-dimensional template has recesses or formations, which are thermoformed or vac-formed, and into which the instruments can be placed.

The template 14 indicates the surgical instruments 100 to be contained within the bag 2 and their relative layout. The layout is indicated graphically, thus clearly assisting the operator to include the correct instruments. This is illustrated schematically in FIG. 3. Alternatively, the layout can be indicated by means of alphanumeric symbols, such as words or codes.

The bag 2 can also contain labels, RFID tracking tags, barcodes, accessories, QRcode, optical markers for virtual reality/augmented reality systems.

Once all of the surgical instruments 100 are placed in the bag 2, the opening 6 is sealed so that all the surgical instruments 100 are contained within the bag 2. This sealing is carried out using heat sealing, although other sealing mechanisms could be used, for example using a zip fastening mechanism 13.

A liquid sterilising agent 15 is then fed into the bag 2 under gravity through the port 7. This is done by connecting a container 16 of sterilising agent by means of a flexible tube 17 to the port 7. A sufficient amount of sterilising agent 15 is fed into the bag 2 to ensure that all of the surgical instruments 100 within the bag 2 are covered with sterilising agent 15. To assist, the bag can be placed on an inclined surface 21 to assist in ensuring that the sterilising agent 15 runs throughout the interior 19 of the bag 2 so as to cover all of the surgical instruments 100. This part of the process is illustrated in FIG. 4.

Alternatively, the bag 2 can be manually manipulated by shaking or other movement of the bag 2 so that the surgical instruments are in contact with the sterilisation agent 15 and such that contact between the sterilising agent 15 and the surfaces of the surgical instruments 100 is maximised.

Once the required amount of sterilising agent 15 has been fed into the bag 2, the, or another, flexible tube 17 is connected between a vacuum pump 18 and the port 7. The vacuum pump 18 is operated to remove air and the majority of the sterilising agent 15 from the interior 19 of the bag 2. In removing the air, a vacuum is formed within the interior 19 and the flexible walls 3, 4 of the bag 2 collapse to restrain the surgical instruments 100 in place. An amount of liquid sterilising agent 15 will remain in the interior 19 of the bag 2.

The term vacuum in this context is used to mean that after the air is extracted from the interior 19 of the bag 2, the interior 19 is at a lower pressure atmospheric pressure and, preferably, significantly lower.

The use of vacuum pumps to remove air from collapsible bags is known in the art and need not be described in any further detail herein except as is relevant to the present invention.

In this way, each of the surgical instruments 100 in the bag 2 remains in contact with a small amount of sterilising agent 15 remaining in the bag 2 and which serves to maintain the sterilisation process, while the provision of a vacuum in the interior 19 of the bag 2 fixes the position and the layout of the surgical instruments 100 within the bag 2.

The flexible tube that forms the port 7 is then removed and the bag 2 is sealed on all four sides to maintain the vacuum within the bag 2 and to keep the sterilising agent 15 surrounding the surgical instruments 100. Preferably, the flexible tube 7 contains a one-way valve, a faucet/tap or finger-operated valve to ensure that the vacuum is maintained within the bag 2. Where the port 7 is formed as an aperture in the seal of the first wall 3 and second wall 4, the aperture is sealed during or immediately after the vacuum had been applied.

Alternatively, the bag 2 can be provided without a port 7 and the opening 6 can be used for inserting the surgical instruments 100 and also for placing the sterilising agent 15 in the bag 2 and for extracting the air.

If required, the bag 2 can be placed inside a second bag (not shown) with a vacuum suction applied to remove the air from between the first bag 2 and the second bag to provide additional robustness and a further layer of protection. This enables aseptic transfer in which one non-sterile nurse cuts open the bag 2 and holds it open and then a second sterile nurse reaches in to retrieve the inner bag which is still sterile. Both bags contain the sterilising agent 15.

The assembled bag 2 can then be stored on a hanging rack or rail 22 or in a cabinet for selection when required.

An operator, such as a heath professional or technician, is able to simply look at the bags 2 hanging on the rail 22 and select the correct bag 2 containing the surgical instruments 100 required for the particular procedure.

The bag 2 is then carried using the carry handle to the operating theatre for use in the procedure.

In use, the bag 2 is opened using sterile scissors and the surgical instruments 100 can be removed and used.

The bag 2 can be reused to store the instruments 100 after use, keeping them together for reprocessing. If the bag 2 contains a template 14, the template 14 can be used as a shadow board to indicate where the instruments are to be placed. Alternatively, the bag can be disposed of.

The sterilising agent can also include a lubricant if required. In addition, the sterilising agent could include an anti-oxidation agent to prevent corrosion of the instruments 100.

The sterilising agent 15 may comprise an agent with anti-microbial properties. The anti-microbial agent may contain one or more of glutaraldehyde, iodine, povidone-iodine, an alcohol (for example ethanol), trichlorophenol, sodium hypochlorite, and 5-chloro-2-(2,4-dichlorophenoxy)phenol (triclosan). The anti-microbial agent may be provided in an aqueous solution. This list is not limiting and other suitable agents could be used.

Additionally, the surgical instruments 100 can be given a final rinse after the bag 2 is opened so as to remove residual chemical sterilising agents. This can be done by running sterile water through the bag 2 through the port 7 or the opening 6.

If an anti-microbial sterilising agent is used, a neutralizing agent can be placed in the bag 2 with the sterilising agent. The neutralizing agent can be any suitable chemical agent which can react with the sterilising agent after a predetermined time delay to neutralize the anti-microbial sterilising agent. For example, this could be an agent that is oxidised in response to contact with the anti-microbial agent to leave a neutral residue. Low concentration hydrogen peroxide can reduce to water and, as such, would be considered self-neutralizing.

In an alternative, the liquid sterilising agent can be replaced at the stage where the air is being extracted from the bag 2 as long as a proportion of the liquid sterilising agent remains in the bag. For example, air or a sterile lubricant could be inserted into the bag. Alternatively, a disinfecting agent could be used as the replacement fluid. An example could be a mixture of phenol and halogenated phenol compounds manufactured under the trade mark “TCP”.

1. A container for a medical device, the container comprising a first wall and a second wall defining an interior of the container, at least one of the first wall and the second wall being flexible, the medical device being housed within the interior of the container, the container having had suction applied to the interior of the container and sealed such that at least one of the first wall or second wall is configured to encase the medical device and wherein the interior of the container has an amount of a liquid sterilizing agent contained therein.

2. A container for a medical device according to claim 1, wherein the container further includes an opening connecting the interior of the container to an exterior of the container through which the medical device can be inserted.

3. A container for a medical device according to claim 2, wherein the container further includes a zip fastener at the opening.

4. A container for a medical device according to claim 2, wherein the opening is configured for applying the suction to the interior of the container.

5. A container for a medical device according to claim 2, wherein the container further includes at least one port connecting the interior of the container to an exterior of the container.

6. A container for a medical device according to claim 5, wherein the at least one port includes a removable tube.

7. A container for a medical device according to claim 5, wherein the at least one port is an aperture formed in the container.

8. A container for a medical device according to claim 5, wherein the first wall and the second wall are sealed together with the at least one port provided in a junction between the sealed first wall and second wall.

9. A container for a medical device according to claim 1, wherein at least one of the first and second walls comprises a transparent or translucent material.

10. A container for a medical device according to claim 1, wherein the first wall and/or the second wall comprise a flexible material having multiple layers.

11. A container for a medical device according to claim 1, wherein the container includes a carry handle.

12. A container for a medical device according to claim 1, wherein the liquid sterilizing agent comprises an agent with microbicidal, and/or fungicidal, and/or virucidal and/or sporicidal properties.

13. A container for a medical device according to claim 1, wherein the medical device is a surgical instrument.

14. A container for a medical device according to claim 13, wherein the container comprises a plurality of surgical instruments.

15. A container for a medical device according to claim 14, wherein the container further includes a template according to which the plurality of surgical instruments are arranged within the container such that the surgical instruments are constrained within the container when sealed and remain in a fixed arrangement relative to each other.

16. A container for a medical device according to claim 15, wherein the template is removably inserted into the interior of the container.

17. A container for a medical device according to claim 1, wherein the medical device is a prosthetic implant.

18. A method of sterilizing a medical device, the method including the steps of:
providing a container for the medical device, the container having a first wall and a second wall defining an interior of the container, at least one of the first wall and second wall being flexible, the container further including an opening connecting the interior of the container to the exterior of the container;
placing the medical device into the interior of the container;
introducing a liquid sterilizing agent into the interior of the container;
extracting air from the container; and
sealing the container.

19. A method of sterilizing a medical device according to claim 18, wherein the medical device is placed into the interior of the container through the opening.

20. A method of sterilizing a medical device according to claim 18, wherein the liquid sterilizing agent is introduced into the container via the opening.

21.-33. (canceled)
